(19) United States
(12) Patent Application Publication
(10) Pub. No.: US 2002/0160045 A1
(43) Pub. Date: Oct. 31, 2002

(54) Therapeutic dosage form for delivering oxybutynin

(76) Inventors: Michael A. Desjardin, Sunnyvale, CA (US); Paul M. Hwang, Mountain View, CA (US); Halle Treanor, San Diego, CA (US)

Correspondence Address: ALZA CORPORATION, P O BOX 7210, INTELLECTUAL PROPERTY DEPARTMENT, MOUNTAIN VIEW, CA, 940397210

(21) Appl. No.: 10/164,138

(22) Filed: Jun. 4, 2002

Related U.S. Application Data
(62) Division of application No. 09/905,527, filed on Jul. 13, 2001.
Application No. 09/905,527 is a continuation of application No. 09/306,702, filed on May 6, 1999, now abandoned.
(60) Provisional application No. 60/088,067, filed on Jun. 5, 1998.

(51) Int. Cl. A61K 9/22; A61K 9/24; A61K 31/24

(52) U.S. Cl. 424/468; 514/540

(57) ABSTRACT

A dosage form, a therapeutic composition, and the use thereof is disclosed for administering a therapeutic agent accompanied by a pharmaceutically acceptable means administered for an indicated therapy.

### RELATED APPLICATIONS

This application claims the benefit of U.S. Provisional Application No. 60/088,067, filed on Jun. 5, 1998.

### FIELD OF THE INVENTION

The present invention pertains to both a novel and useful drug delivery system. More particularly, the invention relates to a sustained release dosage form that delivers the prescribed dose of drug over an extended period of time. The invention concerns also a method of administering the prescribed dose of drug to a patient for producing the intended therapeutic benefit.

### BACKGROUND OF THE INVENTION

Dosage forms for administering a beneficial drug to a biological-fluid environment of use, are known to the medical and veterinary sciences. For example, dosage forms are known in U.S. Pat. No. 3,845,770 issued to Theeuwes and Higuchi, in U.S. Pat. No. 3,916,899 issued to the same patentees, and in U.S. Pat. No. 4,612,008 issued to Wong, Barclay, Deters, and Theeuwes. These patents disclosed a wall that surrounds a composition comprising a dose of drug, and in another embodiment a composition comprising a dose of drug and a hydrophilic polymer carrier. The wall of the dosage forms is permeable to the passage of fluid, and it comprises a passageway for delivering the drug from the dosage form. The dosage forms of these patents are effective for delivering a drug to aqueous environment including biological fluids over time. A pioneering improvement in the above mentioned dosage forms was presented to the pharmaceutical dispensing art by inventor Theeuwes in U.S. Pat. Nos. 4,111,202; 4,111,203; and 4,203,439. In these three patents, the delivery kinetics of the dosage forms was enhanced for delivering drug by incorporating a film-hydrogel-piston arrangement into the dosage form, that pushed the drug from the dosage form over time. A quantum advancement in these dosage forms was made by Cortese and Theeuwes in U.S. Pat. No. 4,327,725 and by Wong, Barclay, Deters and Theeuwes in U.S. Pat. No. 4,612,008. The dosage form disclosed in these patents comprise a beneficial drug formulation and a hydrogel that expands and pushes the drug formulation through a passageway from the dosage form.

Dosage forms for administering a drug to the gastrointestinal tract comprising an environmental fluid are disclosed also in U.S. Pat. No. 5,667,801 issued to Baichwal. The dosage form disclosed in this patent consists of a heteropolysaccharide and a homopolysaccharide capable of cross-linking the heteropolysaccharide when exposed to the environmental fluid. Another dosage form is disclosed in U.S. Pat. No. 4,443,428 issued to Oshlack et al. The dosage form of this patent consists of a hydrated hydroxyalkylcellulose and a hydrophobic higher aliphatic alcohol in a matrix melt granulation controlled release core and a pharmacologically active substance. In U.S. Pat. No. 5,558,879 issued to Chen et al, a dosage form is disclosed consisting of a compressed core of a drug, a water soluble polymer, and a dual coating around the core.

The dosage forms disclosed in the above patents operate for their intended therapy. While these dosage forms are useful, their use often is limited in therapy. For instance, a residual fraction of the drug dose may remain in the dosage form thus preventing a patient from receiving the intended dose. Also, a pharmaceutical carrier used for transporting a drug from the dosage form may be sticky in the presence of fluid that enters the dosage and restrict passage of the drug from the dosage form. Then too, a polymer carrier for transporting the drug may not hydrate and this may lead to the unwanted effects of drug entrapment within the dosage form. In these instance, the patient may not receive the intended therapy.

It will be appreciated by those versed in the drug dispensing arts, that if a dosage form is made available that overcomes the tribulations of the prior art, such a dosage form would have a positive value in the drug dispensing art. Likewise, it will be scientifically self-evident to those versed in the drug delivery art that if a dosage form is made available that delivers essentially the maximum dose, such a dosage form would have immediate acceptance in the fields of human and veterinary medicine.

### OBJECTS OF THE INVENTION

Accordingly, in view of the above presentation, it is an immediate object of this invention to provide a dosage form for the sustained and controlled delivery of a beneficial drug that overcomes the shortcomings associated with the prior art.

Another object of the present invention is to provide a novel dosage form that delivers essentially the preselected and prescribed dose of drug to a patient in need of the drug.

Another object of the invention is to provide a sustained-release dosage form comprising a dose of drug and pharmaceutically-acceptable chemical means for aiding the dosage form in delivering the maximum dose of drug.

Another object of the invention is to provide a sustained-release, controlled-delivery dosage form comprising a dose of drug and a pharmaceutically acceptable drug-delivery means for reducing and/or eliminating the amount of residual-drug retained in the dosage form.

Another object of the present invention is to reduce the drug-delivery start-up time in a dosage form.

Another object of the invention is to provide a dosage form comprising a drug composition comprising a dose of drug, a pharmaceutically acceptable salt and a pharmaceutically acceptable hydrophilic polymer possessing a low-molecular weight, and a push-displacement composition comprising a hydrophilic polymer possessing a higher molecular weight than the hydrophilic polymer in the drug composition whereby the lower drug composition to push composition weight ratio provides a more immediate start-up time for the dosage form to deliver the drug.

Another object of the invention is to provide a therapeutic composition comprising a drug, a pharmaceutically acceptable salt, and a pharmaceutically acceptable polymer carrier for administering a drug orally to a patient for its intended therapy.

Another object of the invention is to provide a therapeutic composition for delivering a beneficial drug to be administered as the composition, or for incorporating the composition into a dosage form, which composition in either application comprises a drug, a pharmaceutically acceptable salt, and a pharmaceutically acceptable surfactant which pharmaceutically acceptable salt and the pharmaceutically acceptable surfactant improves the amount of drug delivered by reducing the residual drug remaining in the composition and in the dosage form after twenty-four hours of drug delivery.

Another object of the invention is to provide a method for administering essentially a complete dose of drug to a patient by administering the drug using the dosage form and/or the drug composition provided by this invention.

Another object of the invention is to make available a composition of matter comprising chemical means for providing and for maintaining a high level of osmotic activity for use in delivering a beneficial drug orally to a patient in need of drug therapy.

Another object of the invention is to provide a dosage form for delivering in vivo a beneficial drug that is difficult to deliver and now can be delivered by this invention in a therapeutically effective dose over twenty-four hours.

Another object of the invention is to provide a dosage form manufactured as a pharmaceutically acceptable controlled-release oral tablet comprising a single composition possessing osmotic properties and can be manufactured by conventional compression and coating techniques.

Another object of the invention is to provide a method for administering a pharmaceutically active drug over twenty-four hours from an initially solid pharmaceutically acceptable dosage form comprising a pharmaceutically acceptable salt of the pharmaceutically active drug, a different pharmaceutically acceptable salt, and a pharmaceutically acceptable surfactant for administering the drug orally to a patient.

Other objects, features, aspects, and advantages of the invention will be more apparent to those versed in the dispensing arts from the following detailed specification and the accompanying claims.

### DETAILED DISCLOSURE OF THE INVENTION

The term drug, as used herein, denotes a therapeutically active drug, including any physiologically or pharmacologically active substance that produces a local, or a systemic effect in animals, including humans. The terms physiologically and pharmacologically are defined in Stedman's Medical Dictionary, (1966), published by Williams and Wilkins, Baltimore, Md. The active drug include inorganic and organic drugs that act on the central nervous system, depressants, hypnotics, sedatives, psychic energizers, tranquilizers, anticonvulsants, muscle relaxants, anti-Parkinsons, analgesic, anti-inflammatories, local anesthetics, muscle contractants, anti-microbials, anti-malarials, hormones, contraceptives, diuretics, sympathomimetics, paraciticides, neoplastics, hypoglycemics, ophthalmics, electrolytes, and cardiovascular drugs. These drugs are known in Pharmaceutical Sciences, edited by Remington, 16th Ed., (1980), published by Mack Publishing Company, Easton, Pa.

The present invention delivers, in one manufacturer, a drug selected from the group consisting of oxybutynin and its pharmaceutically acceptable salt. The dose of oxybutynin in a therapeutic composition administered as the base or the dose of oxybutynin salt in a therapeutic composition in a dosage form is, in both manufacturers, 240 ng to 650 mg (nanogram to milligram) or expressed as weight percent (wt %), 2 wt % to 25 wt %. The oxybutynin pharmaceutically acceptable salt comprises a member selected from the group consisting of acetate, bitartrate, citrate, edetate, edisylate, estolate, esylate, fumarate, glyceptate, gluconate, glutamate, hydrobromide, hydrochloride, hydroiodide, lactate, malate, maleate, mandelate, mesylate, methylnitrate, museate, napsylate, nitrate, pamoate, pantothenate, phosphate, salicylate, stearate, succinate, sulfate, tannate, and tartrate. The drug oxybutynin can be present as the base, as the salt, as the racemate, as the R-enantiomer, and as the S-enantiomer.

The therapeutic composition used for delivering the drug, and used for manufacturing a sustained-release dosage form comprises a pharmaceutically acceptable hydrophilic polymer. Representative of a hydrophilic polymer is polyalkylene oxide. The polyalkylene oxide polymers comprise polyethylene oxide of 100,000 weight-average molecular weight, polyethylene oxide of 200,000 weight-average molecular weight, polyethylene oxide of 300,000 weight-average molecular weight, a blend of polyethylene oxide of 100,000 weight-average molecular weight and a polyethylene oxide of 200,000 weight-average molecular weight in a blend of 1 wt % to 99 wt % to 99 wt % to 1 wt %, polypropylene oxide of 150,000 weight-average molecular weight, and a blend of polyethylene oxide and polypropylene oxide. The therapeutic composition comprises 10 mg to 250 mg of the pharmaceutically acceptable hydrophilic polymer. The polymers are availably commercially from the Union Carbide Corporation, Danbury, Conn.

The therapeutic composition used as the therapeutic composition and for providing a dosage form comprises 0 mg to 50 mg of a binder, and in a manufactured embodiment from 0.5 mg to 50 mg of the binder. Representative of non-toxic binders comprise a member selected from the group consisting of acacia, alginic acid, Carbomer® polymer consisting of acrylic acid cross-linked with allylsucrose or allyl ethers of pentaerythriol, dextrin, gelatin, guar gum, maltodextrin, polyvinylpyrrolidone, pregelatinized starch, sodium alginate, starch, and zein. The binders include hydroxypropylalkylcellulose of 9,000 to 150,000 average-number molecular weight selected from the group consisting of hydroxypropylmethylcellulose, hydroxypropylethylcellulose, hydroxypropylbutylcellulose, and hydroxypropylpentylcellulose. The binder imparts cohesive qualities to the composition.

The therapeutic composition used for providing the therapeutic composition and for providing a dosage form comprises 0 mg to 45 mg, and in present embodiments from 1 mg to 45 mg of a therapeutically active salt selected from the group consisting of inorganic and organic salts. Representative salts comprise a member selected from the group consisting of sodium chloride, potassium chloride, potassium acid phosphate, sodium citrate, potassium edelate, magnesium sulfate, magnesium chloride, lithium sulfate, potassium sulfate, sodium tartarate, potassium citrate, potassium fumarate, sodium lysinate, potassium succinate, and sodium glycinate.

The therapeutic composition comprises a lubricant used during manufacture to prevent the composition sticking to the walls or punch face of manufacturing equipment. The concentration of lubricant is 0.00 mg to 10 mg and usually 0.01 mg to 10 mg. Typical lubricants include magnesium stearate, sodium stearate, stearic acid, calcium stearate, magnesium oleate, oleic acid, potassium oleate, sodium oleate, caprylic acid, sodium stearyl fumarate, magnesium palmitate, salts of fatty acids, salts of alicyclic acids, salts of aromatic acids, and a mixture of a fatty, alicyclic or aromatic acid, and fatty, alicyclic or aromatic acid blend.

The therapeutic composition and the dosage form containing the therapeutic composition comprise a surfactant. The surfactant functions to increase the water solubility of constituents in the therapeutic composition, the surfactant reduces interfacial tension between constituents, the surfactants enhances the free-flow and delivery of constituents, and the surfactant lessens the incidence of constituent retention in a dosage form. The surfactants useful for the purpose of this invention comprise amphoteric surfactants, anionic surfactants, cationic surfactants and nonionic surfactants. The therapeutic composition and the dosage form of this invention comprise a nonionic surfactant such as polyoxyethylenated sorbitol monolaurate comprising 20 moles of ethylene oxide available as Tween® 20, polyoxyethylenated sorbitan monopalmitate comprising 20 moles of ethylene oxide commercially available as Tween 40, polyoxyethylenated sorbitan monostearate comprising 20 moles of ethylene oxide commercially available as Tween 60, polyoxyethylenated sorbitan monostearate comprising 4 moles of ethylene oxide commercially available as Tween 61, polyoxyethylenated sorbitan tristearate comprising 20 moles of ethylene oxide available as Tween 65, polyoxyethylenated sorbitan monooleate comprising 20 moles of ethylene oxide available as Tween 80, polyoxyethylenated sorbitan trioleate containing 20 moles of ethylene oxide available as Tween 85, and polyoxyethylenated stearic acid comprising 8 moles of ethylene oxide available as Myrj® 45. The surfactants are available from Atlas Chemical Industries, Wilmington, Del. The concentration of surfactant in a therapeutic composition is 0.01 mg to 25 mg, in operation 0.01 mg to 5 mg, or 1 wt % to 7.5 wt %.

The therapeutic composition can comprise a colorant for identifying the drug contained therein. The colorant comprises 0.00 mg to 4.5 mg of FD&C Red No. 3; FD&C Red No. 40; FD&C Yellow No. 5; FD&C Yellow No. 6; FD&C Blue No. 1; FD&C Blue No. 2; FD&C Green No. 3; iron oxides including red ferric oxide and yellow ferric oxide; titanium dioxide; acid fuchsine; and allure red.

The dosage form provided by the invention in an additional embodiment comprises a wall that surrounds the therapeutic composition. The wall comprises an exit passageway to provide for the continuous release of drug. The dosage form of the invention is a sustained-release dosage form as the dosage form provides for the prolonged and extended duration of drug delivery over time achieved by conventional drug delivery forms such as tablets and capsules. The sustained-release dosage form provided controlled delivery over 24 hours, wherein the controlled-rate of delivery is provided by the dosage form.

The wall that surrounds the therapeutic drug composition comprises totally, or in at least a part a semipermeable composition. The semipermeable composition is permeable to the passage of an aqueous fluid, or a biological fluid present in the gastrointestinal tract, and it is impermeable to the passage of drug. The wall is nontoxic and it maintains its physical and chemical integrity during the dispensing time of a drug. The phrase, maintains its physical and chemical integrity means the wall does not lose its structure during the dispensing of a drug. The wall comprises a composition that does not adversely affect an animal, a human, or components of the dosage form. Compositions for forming the wall are, in one embodiments, comprised of a member selected from the group consisting of a cellulose ester polymer, a cellulose ether polymer and a cellulose ester-ether polymer. These cellulosic polymers have a degree of substitution, DS, on the anhydroglucose unit, from greater than 0 up to 3 inclusive. By “degree of substitution” is meant the average number of hydroxyl groups originally present on the anhydroglucose unit comprising the cellulose polymer that are replaced by a substituting group. Representative of wall polymers comprise a member selected from the group consisting of cellulose acylate, cellulose diacylate, cellulose triacylate, cellulose acetate, cellulose diacetate, cellulose is triacetate, mono-, di- and tricellulose alkanylates, mono, di-, and trialkenylates, mono-, di- and tricellulose alkinylates, and mono-, di- and triaroylates. Exemplary polymers include cellulose acetate having a DS of up to 1 and an acetyl content of up to 31%; cellulose acetate having a DS of 1 to 2 and any acetyl content of 21 to 35%; cellulose acetate having a DS of 2 to 3 and an acetyl content of 35 to 44.8%, and the like. More specific cellulosic polymers comprise cellulose propionate having a DS of 1.8, a propyl content of 39.2 to 45% and a hydroxyl content of 2.8 to 5.4; cellulose acetate butyrate having a DS of 1.8, an acetyl content of 13 to 15% and a butyl content of 34 to 39%; cellulose acetate butyrate having a acetyl content of 2 to 29%, a butyl content of 17% to 53% and a hydroxy content of 0.5 to 4.7; cellulose triacylates having a DS of 2.9 to 3, such as cellulose trivalearate, cellulose trilaurate, cellulose tripalmitate, cellulose trisuccinate and cellulose trioctanoate; celluloses diacylate having a DS of 2.2 to 2.6, such as cellulose disuccinate, cellulose dipalmitate, cellulose dioctanoate, cellulose dipentanoate, co-esters of cellulose, such as cellulose acetate butyrate, and cellulose acetate propionate.

Additional semipermeable polymers for providing a wall that surrounds a therapeutic composition comprise acetaldehyde dimethylcellulose acetate; cellulose acetate ethylcarbamate; cellulose acetate methylcarbamate; cellulose diacetate propylcarbamate; cellulose acetate diethylaminoacetate; semipermeable polyamide; semipermeable polyurethane; semipermeable sulfonated polystyrene; semipermeable crosslinked selective polymer formed by the coprecipitation of a polyanion and polycation, as disclosed in U.S. Pat. Nos. 3,173,876; 3,276,586; 3,541,005; 3,541,006 and 3,546,876; semipermeable polymers as disclosed by Loeb and Sourirajan in U.S. Pat. No. 3,133,132; semipermeable, lightly crosslinked polystyrenes; semipermeable crosslinked poly (sodium styrene sulfonate); semipermeable cross-linked poly (vinylbenzyltrimethyl ammonium chloride); and semipermeable polymers possessing a fluid permeability of 2.5×10⁻⁸ to 5×10⁻² (cm²/hr·atm), expressed per atmosphere of hydrostatic or osmotic pressure difference across the semipermeable wall. The polymers are known to the polymer art in U.S. Pat. Nos. 3,845,770; 3,916,899 and 4,160,020; and in Handbook of Common Polymers, Scott, J. R. and W. J. Roff, 1971, CRC Press, Cleveland, Ohio.

The present invention provides additionally a sustained-release dosage form comprising a wall that surrounds a therapeutic drug composition and a push-displacement composition. The wall comprising a passageway and the therapeutic drug composition were presented above and that presentation is incorporated into this disclosure of the dosage form comprising the therapeutic and push-displacement compositions. In this dosage form, the therapeutic composition is initially in contact with the push-displacement composition. The therapeutic composition and the push-displacement composition operate together as a matrix to provide therapy. The push-displacement composition comprises 10 mg to 350 mg of a pharmaceutically-acceptable hydrophilic polymer that imbibes fluid through the wall, causing it to expand and push-displace the therapeutic composition through an exit from the dosage form. Representative of a hydrophilic polymer comprises a member selected from the group consisting of a polyalkylene oxide of 1,000,000 to 8,000,000 weight-average molecular polyethylene oxide of 1,000,000 weight-average molecular weight, polyethylene oxide of 5,000,000 weight-average molecular weight, polyethylene oxide of 7,500,000 weight-average molecular weight, polypropylene oxide of 2,000,000 weight-average molecular weight, and polypropylene oxide of 4,000,000 weight-average molecular weight. The hydrophilic polymer comprises 20 mg to 250 mg of an alkali carboxymethylcellulose of 10,000 to 6,000,000 weight-average molecular weight such as sodium carboxymethylcellulose or potassium carboxymethylcellulose.

The push-displacement composition comprises compositional-forming ingredients represented by 0.00 mg to 250 mg of a hydroxyalkylcellulose of 7,500 to 2,500,000 weight-average molecular weight represented by a member selected from the group consisting of hydroxymethylcellulose, hydroethylcellulose, hydroxypropylcellulose, hydroxybutylcellulose, and hydroxypentylcellulose. The push-displacement composition comprises 1 mg to 60 mg of an osmagent selected from the group consisting of sodium chloride, potassium chloride, potassium acid phosphate, tartaric acid, citric acid, raffinose, magnesium sulfate, magnesium chloride, urea, inositol, sucrose, glucose and sorbitol. The push-displacement composition comprises 0.1 mg to 30 mg of a hydroxypropylalkylcellulose of 9,000 to 225,000 average-number molecular weight, selected from the group consisting of hydroxypropylethylcellulose, hydroxypropylpentylcellulose, hydroxypropylmethylcellulose, and hydropropylbutylcellulose. The push-displacement composition can comprise 0.00 to 1.5 mg of an antioxidant selected from the group consisting of ascorbic acid, butylated hydroxyanisole, butylatedhydroxyquinone, butylhydroxyanisol, hydroxycomarin, butylated hydroxytoluene, cephalm, ethyl gallate, propyl gallate, octyl gallate, lauryl gallate, propylhydroxybenzoate, trihydroxybutylrophenone, dimethylphenol, diterlbutylphenol, vitamin E, lecithin and ethanolamine. The push-displacement composition comprises 0.1 mg to 7 mg of a lubricant selected from the group consisting of calcium stearate, magnesium stearate, zinc stearate, magnesium oleate, calcium palmitate, sodium suberate, potassium laureate, salts of fatty acids, salts of alicyclic acids, salts of aromatic acids, stearic acid, oleic acid, palmitic acid, a mixture of a salt of a fatty, alicyclic or aromatic acid, and a fatty, alicyclic or aromatic acid, with the total weight of all ingredients in the push-displacement composition equal to 100 wt %.

The expression “passageway” as used herein comprises means and methods suitable for the metered release of the therapeutic drug from the compartment of the dosage form. The exit means comprises at least one passageway, including orifice, bore, aperture, pore, porous element, hollow fiber, capillary tube, porous overlay, or porous element that provides for the osmotic controlled release of oxybutynin. The passageway includes a material that erodes or is leached from the wall in a fluid environment of use to produce at least one dimensioned passageway. Representative materials suitable for forming a passageway, or a multiplicity of passageways comprise a leachable poly(glycolic) acid or poly(lactic) acid polymer in the wall, a gelatinous filament, poly(vinyl alcohol), leachable polysaccharides, salts and oxides. A pore passageway, or more than one pore passageway, can be formed by leaching a leachable compound, such as sorbitol, from the wall. The passageway possesses controlled-release dimensions, such as round, triangular, square and elliptical, for the metered release of oxybutynin from the dosage form. The dosage form can be constructed with one or more passageways in spaced apart relationship on a single surface or on more than one surface of the wall. The expression “fluid environment” denotes an aqueous or biological fluid as in a human patient, including the gastrointestinal tract. Passageways and equipment for forming passageways are disclosed in U.S. Pat. Nos. 3,845,770; 3,916,899; 4,063,064; 4,088,864 and 4,816,263. Passageways formed by leaching are disclosed in U.S. Pat. Nos. 4,200,098 and 4,285,987.

### DESCRIPTION FOR MANUFACTURING THE INVENTION

The wall of the dosage form can be formed by using the air suspension procedure. This procedure consists in suspending and tumbling the composition or the layers in a current of air and wall-forming composition until a wall is applied to the therapeutic composition, or is applied to the therapeutic composition and push-displacement composition matrix. An air suspension procedure is well suited for independently forming the wall. The air suspension procedure is described in U.S. Pat. No. 2,799,241; J. Am. Pharm. Assoc., Vol. 48, pp. 451-459 (1959); and ibid, Vol. 49, pp. 82-84 (1960). The wall can be formed with a wall-forming composition in a Wurster® air suspension coater using an organic solvent, such as acetone-water cosolvent 90:10 (wt:wt) with 2.5 wt % to 7 wt % polymer solids. An Aeromatic® air suspension coater using, for example, a methylene dichloride-methanol cosolvent comprising 87:13 (v:v) can be used for applying the wall. Other wall-forming techniques, such as pan coating system, wall forming compositions are deposited by successive spraying of the composition or the bilayered arrangement, accompanied by tumbling in a rotating pan. A larger volume of cosolvent can be used to reduce the concentration of polymer solids to produce a thinner wall. Finally, the wall of the coated compartments are laser or mechanically drilled, and then dried in a forced air or humidity oven for 1 to 3 days or longer to free the solvent. Generally, the walls formed by these techniques have a thickness of 2 to 20 mils (0.051 to 0.510 mm) with a preferred thickness of 2 to 6 mils (0.051 to 0.150 mm).

The dosage form of the invention is manufactured by standard manufacturing techniques. For example, in one manufacture the beneficial drug and other ingredients comprising a therapeutic composition or comprising a first compositional layer facing the exit means are blended, or they are blended then pressed, into a solid layer. The drug and other ingredients can be blended with a solvent and formed into a solid or semisolid formed by conventional methods such as ball-milling, calendaring, stirring or roll-milling and then pressed into a selected shape. The composition posses dimensions that correspond to the internal dimensions of the area the composition is to occupy in the dosage form. In a dosage form comprising two separate but contacting compositions in a bilayer arrangement, the bilayer composition possess dimensions corresponding to the internal lumen of the dosage form. The layering of the drug composition and the push-displacement composition can be fabricated by conventional press-layering techniques. The compositions are compressed and then surrounded with an outer wall. A passageway is drilled, by laser or mechanically through the wall to contact the therapeutic composition for releasing the drug from the dosage form. The dosage form is optically oriented automatically by the drilling equipment for forming an exit passageway on the preselected drug surface.

In another manufacture, the dosage form is manufactured by a granulation technique. Granulation is defined in the Encyclopedia of Pharmaceutical Technology, edited by Swarbrich and Boylan, as a process of size enlargement in which the original particle can still be identified, pp. 121-127, 393-400, and 423-446 (1991). One granulation procedure is the wet granulation. In the wet granulation technique the oxybutynin and the ingredients comprising the first layer are blended using an organic or inorganic solvent, such as isopropyl alcohol-methylene dichloride 80:20 (v:v) as the granulation fluid. Other granulating fluid, such as water; isopropyl alcohol, or denatured alcohol 100% can be used for this purpose. The ingredients forming the first layer are individually passed through a 40 mesh screen and then thoroughly blended in a mixer. Next, other ingredients comprising the therapeutic composition are dissolved in a portion of the granulation fluid, such as the cosolvent described above. Then, the latter prepared wet blend is slowly added to the drug blend with continual mixing in the blender. The granulating fluid is added until a wet blend mass is produced, which wet mass is then forced through a 20 mesh screen onto oven trays. The blend is dried for 18 to 24 hours at 25° C. to 40° C. The dry granules are then screened with a 16 mesh screen. Next, a lubricant is passed through an 60 mesh screen and added to the dry screened granule blend. The granulation is put into milling jars and mixed on a jar mill for 2 to 10 minutes. The first and second compositions are pressed into a layered tablet, for example, in a Manesty® layer press.

Another manufacturing process that can be used for providing the drug and hydrogel compositions comprises blending their powdered ingredients in a fluid bed granulator. After the powdered ingredients are dry blended in the granulator, a granulating fluid, for example, poly(vinylpyrrolidone) in a solvent, such as in water, is sprayed onto the respective powders. The coated powders are then dried in a granulator. This process coats the ingredients present therein while spraying the granulating fluid. After the granules are dried, a lubricant, such as stearic acid or magnesium stearate, is blended as above into the mixture. The granules are then pressed in the manner described above. In another embodiment, when the fluid bed granulating process is used to manufacture the hydrogel layer, the antioxidant present in the polyalkylene oxide can be removed during the processing step. If antioxidant is desired it can be added to the hydrogel formulation; this can be accomplished during the fluid bed granulation described above.

The dosage form of this invention is manufactured in another embodiment by mixing the drug with composition-forming ingredients and pressing the composition into a solid composition possessing dimensions that correspond to the internal dimensions of the dosage form adjacent to a passageway. In another embodiment, the drug and other drug composition forming ingredients and a solvent are mixed into a solid, or semi-solid, by conventional methods such as ball-milling, calendaring, stirring or roll-milling, and then pressed into a preselected, layer-forming shape.

In the manufactures as presented above, the manufacture comprising a composition or comprising a layer of a composition comprising a hydrogel osmopolymer and an optional osmagent are placed in contact with the layer comprising the drug oxybutynin, and the two layers comprising the layers are surrounded with a semipermeable wall. The layering of the first drug composition and the second hydrogel osmopolymer and optional osmagent composition can be accomplished by using a conventional two-layer tablet press technique. The wall can be applied by molding, spraying or dipping the pressed shapes into wall-forming materials. Another technique that can be used for applying the wall is the air suspension coating procedure. This procedure consists in suspending and tumbling the two layers in a current of air until the wall forming composition surrounds the layers. Manufacturing procedures are described in Modern Plastics Encyclopedia, Vol. 46, pp. 62-70 (1969); and in Pharmaceutical Sciences, by Remington, 14th Ed., pp. 1626-1948 (1970), published by Mack Publishing Co., Easton, Pa. The dosage form can be manufactured by following the teaching in U.S. Pat. Nos. 4,327,725; 4,612,008; 4,783,337; 4,863,456; and 4,902,514.

Exemplary solvents suitable for manufacturing the wall, the compositions and the dosage form include inert inorganic and organic solvents that do not adversely harm the materials, the wall, the layer, the composition and the drug wall. The solvents broadly include members selected from the group consisting of aqueous solvents, alcohols, ketones, esters, ethers, aliphatic hydrocarbons, halogenated solvents, cycloaliphatics, aromatics, heterocyclic solvents and mixtures thereof. Typical solvents include acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethylacetate, methylene dichloride, ethylene dichloride, propylene dichloride, carbon chloroform, nitroethane, nitropropane, tetrachloroethane, ethyl ether, isopropyl ether, cyclohexane, cyclo-octane, toluene, naphtha, 1,4-dioxane, tetrahydrofuran, diglyme, aqueous and nonaqueous mixtures thereof, such as acetone and water, acetone and methanol, acetone and ethyl alcohol, methylene dichloride and methanol, and ethylene dichloride and methanol.

The release of drug from a therapeutic composition, from a dosage form, or the dissolution of a drug from a therapeutic composition or dosage form indicates the drug entering into solution upon its delivery as provided by this invention is measured by the following procedure. First, a drug receiving solution, such as, gastrointestinal fluid, such as simulated gastric fluid, simulated intestinal fluid, hydrochloride acid, or a base, is used as the dissolution media. Formulas for preparing simulated gastric fluid and simulated intestinal fluid are specified in The United States Pharmacopea 23, pp. 2053 (1995). A dosage form is placed into the dissolution media is sampled at a constant time interval over the time period of the dissolution. The filtered samples are assayed by a reversed high pressure liquid chromatography with detection by UV. The concentration of the samples is measured against a standard curve containing, for example, at least five standard points. Procedures for dissolution testing are reported in The United States Pharmacopoeia, The National Formulary, pp. 1791-1796 (1995); Pharmaceutical Sciences, by Remington, 17th Ed., pp. 653-666 (1985); and USP XXII, Dissolution Paddle Analysis, pp. 1578-1579 (1990).

The release rate of drug from a dosage form manufactured by this invention can be ascertained by the following procedure. The procedure comprises placing the dosage form in the aqueous test media, with rotational stirring of the USP paddle of 50 to 200 rpm, and taking aliquots of the release rate solution, followed by their injection into a chromatographic system to quantify the amount of drug released during specified test intervals. The drug, for example, is resolved on a column and detected by UV absorption. Quantitation is performed by linear regression analysis of peak areas from a standard curve containing at least five standard points.

An alternative method of measuring release performance of the dosage form comprises attaching a dosage form to a plastic rod with the orifice exposed to the drug receiving solution. Then, attaching the rod to a release arm, with the arm affixed to an up/down reciprocating shaker, which operates at an amplitude of about 3 cm and 2 seconds per cycle. Then, continuously immersing the dosage form in 50 ml test tubes containing 30 ml of H₂O equilibrated in a constant temperature water bath at 37° C.±0.5° C. Next, at the end of each interval, transfer the dosage form to the next row of new test tubes containing a receiving solution, such as water. After the release pattern is complete, remove the tubes and allow to cool to room temperature, followed by filling the calibrated tubes to the 50 ml mark with a solvent, such as acetone. The samples are mixed immediately, transferred to sample vials, followed by chromatography analysis. Another method comprises placing the dosage form in a basket that is immersed repeatedly in the receiving solution, with the complete performance of the test as described in this paragraph.

### EXAMPLES PROVIDED BY THE INVENTION

The following examples are merely illustrative of the present invention and they should not be considered as limiting the scope of the invention in any way, as these examples and other equivalents thereof will become apparent to those versed in the art in the light of the present disclosure and the accompanying claims.

### Example 1

A therapeutic composition comprising oxybutynin hydrochloride provided by the invention is prepared as follows: first, 103 grams of oxybutynin hydrochloride is dissolved in 1200 ml (milliliters) of anhydrous ethanol. Separately, 2,280 g of polyethylene of 200,000 weight-average molecular weight, 150 g of hydroxypropylmethylcellulose of 9,200 average-number molecular weight and 450 g of sodium chloride are dry blended in a conventional blender for 10 minutes to yield a homogenous blend. Next, the oxybutynin ethanol solution is added slowly to the blend, with the blender continuously blending until all the ingredients are added to the three component dry blend, with the blending continued for another 8 to 10 minutes. The blended wet composition is passed through a 16 mesh screen and dried overnight at a room temperature of 72° F. (22.2° C.). Then, the dry granules are passed through a 20 mesh screen 18 g of magnesium stearate is added, and all the ingredients are ready for formulation into a therapeutic oxybutynin composition. The therapeutic composition comprises 3.4 wt % oxybutynin hydrochloride, 76 wt % polyethylene oxide of 200,000 weight-average molecular weight, 5 wt % hydroxypropylmethylcellulose of 9,200 average-number molecular weight, 15 wt % sodium chloride, and 0.6 wt % magnesium stearate. The therapeutic composition can be administered as the therapeutic composition for its intended oxybutynin therapy. The oxybutynin exhibits antispasmodic activity and it can be used for the management of bladder instability associated with incontinence, often referred to as overactive bladder.

### Example 2

A therapeutic composition comprising oxybutynin is prepared according to Example 1, wherein the therapeutic composition comprises 3.4 wt % oxybutynin hydrochloride, 75 wt % polyethylene oxide of 200,000 weight-average molecular weight, 1 wt % polyoxyethylene sorbitan mono-oleate comprising 20 moles of ethylene oxide, 5 wt % hydroxypropylmethylcellulose of 9,200 average-number molecular weight, 15 wt % sodium chloride, and 0.6 wt % magnesium stearate, for administering oxybutynin over twenty four hours for the nonsurgical treatment of urge incontinence in a patient in need of therapy.

### Example 3

A therapeutic composition for the extended and controlled delivery of oxybutynin is prepared by following the procedure of Example 1. The therapeutic comprises 3.4 wt % of oxybutynin or 3.4 wt % oxybutynin pharmaceutically acceptable salt, a pharmaceutically acceptable carrier comprising 75 wt % polyethylene oxide of 100,000 weight-average molecular weight, 1 wt % polyoxyethylene sorbitan monolaurate comprising 20 moles of ethylene oxide, 5 wt % hydroxypropylethylcellulose of 11,200 average-number molecular weight, 15 wt % sodium citrate, and 0.6 wt % magnesium oleate. The therapeutic composition provides a sustained-release dose profile for treating urge incontinence in a patient.

### Example 4

A sustained-release dosage form is provided by the invention as follows: first, a push-displacement composition is prepared comprising 1274 g of polyethylene oxide of 7,500,000 weight-average molecular weight, 600 g of sodium chloride, and 20 g of ferric oxide are separately screened through a 40 mesh screen. Then, all the ingredients are mixed with 100 g of hydroxypropylmethylcellulose of 11,200 average-number molecular weight to produce a homogenous blend. Next, 300 ml of denatured anhydrous alcohol is added slowly to the blend with continuous mixing for 5 minutes. Then, 1.6 g of butylated hydroxytoluene is added, followed by more blending, with 5 g of magnesium stearate added with 5 minutes of blending, to yield a homogenous blend. The freshly prepared granulation is passed through a 20 mesh screen and allowed to dry for 20 hours at 22.2° C. The push-displacement produced comprises 63.67 wt % polyethylene oxide of 7,500,000 weight-average molecular weight, 30 wt % sodium chloride, 1 wt % ferric oxide, 5 mg hydroxypropylmethylcellulose of 11,200 average-number molecular weight, 0.08 wt % butylated hydroxytoluene, and 0.25 mg of magnesium stearate.

### Example 5

A medical device with a sustained-release profile is prepared as follows: first, 147 mg of the oxybutynin composition of Example 2 is added to a punch die set and tamped. Then, 98 mg of the push-displacement composition of Example 3 is added and the two layers compressed under a pressure head of 1.0 ton (907.18 kg) into a 11/32 inch (0.873 cm) diameter, contacting intimate bilayer matrix.

Next, the bilayered matrix is converted into a medical device as follows: first, a semipermeable wall-forming composition is prepared comprising 95 wt % cellulose acetate having a 39.8% acetyl content and 5 wt % polyethylene glycol having a number-average molecular weight of 3,350 by dissolving the ingredients in a cosolvent comprising acetone and water in 90:10, wt:wt, composition to make 4% solid solution. The wall-forming composition is sprayed onto and around the bilayered matrix.

Next, the semipermeable walled, bilayered matrix is drilled to provide a 20 mil (0.51 mm) orifice to contact the oxybutynin. The residual solvent is removed by drying for 48 hours at 50° C. and 50% relative humidity. Next, the medical devices are dried further for 1 hour at 50° C. to remove excess moisture.

The medical device provided by this example comprises a therapeutic composition comprising 3.4 wt % to 75 wt % polyethylene oxide of 200,000 weight-average molecular weight, 1 wt % polyoxyethylene sorbitan monooleate containing 20 moles of ethylene oxide, 5 wt % hydroxypropylmethylcellulose of 9,200 average-number molecular weight, 0.6 wt % magnesium stearate, and 15 wt % sodium chloride. A push-displacement composition comprising 63.67 wt % polyethylene oxide of 7,500,000 weight-average molecular weight, 30 wt % sodium chloride, 1 wt % ferric chloride, 5 wt % hydroxypropylmethylcellulose of 9,200 average-number molecular weight, 0.08 wt % butylated hydroxytoluene, and 0.25 wt % magnesium stearate. The semipermeable wall comprises 95 wt % cellulose acetate comprising 39.8% acetyl content, and 5 wt % polyethylene glycol of 3,350 number-average molecular weight. The medical device comprises an exit passageway of 20 mils (0.50 mm). The medical device had a start-up delivery time of 1.57 hours and delivered 91.6% of oxybutynin. A medical device lacking the nonionic surfactant exhibited a start-up time of 1.86 hours and delivered 89.8% of its drug. The medical device provided by the invention comprising the surfactant unexpectedly had an earlier start-up time by decreasing the start-up time 0.29 hours for providing earlier therapy, and the same medical device oxybutynin an additional 1.8 hours the equivalent to 0.47 mg more oxybutynin therapy.

### Example 6

A dosage form is provided by following the above examples, wherein the therapeutic composition comprises: (a) 5 mg of oxybutynin hydrochloride, 111.6 mg of polyethylene oxide, 7.35 mg of hydroxypropylmethylcellulose, 1.2 mg of polyoxyethylene sorbitan monolaurate with 20 mol of ethylene oxide, 0.88 mg of magnesium stearate, 22.05 mg of sodium chloride, and 0.12 mg of butylated hydroxytoluene; a wall that surrounds the therapeutic composition permeable to fluid and impermeable to oxybutynin, and an exit in the wall for delivering the oxybutynin.

### Example 7

A medical device manufactured as an oral dosage form is provided according to the present disclosure, wherein the therapeutic composition comprises 10 mg of oxybutynin hydrochloride, 74.8 mg of polyethylene oxide, 1.88 mg of hydroxypropylmethylcellulose, 1.5 mg of polyoxyethylene sorbitan monostearate with 20 mol of ethylene oxide, 0.24 mg of magnesium stearate, 7.05 mg of sodium chloride, and 0.07 mg of butylated hydroxytoluene; a semipermeable wall that surrounds the internal composition said semipermeable wall permeable to fluid flux and impermeable to oxybutynin flux; and a passageway in the wall for delivering the oxybutynin to a patient with acute urinary incontinence, or chronic urinary incontinence.

### Example 8

A medical device designed, shaped and adapted as an oral dosage form tablet is prepared according to the mode and the manner of the invention, wherein the medical device comprises a therapeutic drug core comprising 15 mg of oxybutynin hydrochloride, 72.07 mg of polyethylene oxide, 1.88 mg of hydroxypropylmethylcellulose, 1.75 mg of polyoxyethylene oxide sorbitan mono-oleate with 20 moles of ethylene oxide, 0.23 mg of magnesium stearate, 4.7 mg of sodium chloride, and 0.08 mg of butylated hydroxytoluene; a semipermeable wall that surrounds the drug core for comprising an exit for administering the oxybutynin for treating urge incontinence in a patient.

### Example 9

Medical devices sized, shaped and adapted as an oral dosage form are manufactured according to the invention to provide the following: (1) a therapeutic composition comprising 5.3 wt % oxybutynin, 82.37 wt % polyethylene of 200,000 molecular weight, 2 wt % hydroxypropylmethylcellulose of 9,200 molecular weight, 1 wt % polyoxyethylene sorbitan monooleate with 20 mols of ethylene oxide, 0.25 wt % magnesium stearate, 9 wt % sodium chloride, and 0.08 wt % butylated hydroxytoluene; (2) a therapeutic composition comprising 10.6 wt % oxybutynin hydrochloride, 78.57 wt % polyethylene oxide of 200,000 molecular weight, 1 wt % polyoxyethylene sorbitan mono-oleate with 20 mols of ethylene oxide, 2 wt % hydroxypropylmethylcellulose of 9,200 molecular weight, 0.25 wt % magnesium stearate, 7.5 wt % sodium chloride, and 0.08 wt % butylated hydroxytoluene; and, (3) a therapeutic composition comprising 16 wt % oxybutynin hydrochloride, 76.67 wt % polyethylene oxide of 200,000 molecular weight, 1 wt % hydroxypropylmethylcellulose of 9,200 molecular weight, 1 wt % polyoxyethylene sorbitan mono-oleate with 20 mols of ethylene oxide, 0.25 wt % magnesium stearate, 5 wt % sodium chloride, and 0.08 wt % butylated hydroxytoluene; which therapeutic compositions (1), (2), and (3) independently are in laminated arrangement with (4) a push-displacement composition comprising 63.37 wt % of polyethylene oxide of 2,000,000 molecular weight, 30 wt % sodium chloride, 5 wt % hydroxypropylmethylcellulose of 9,200 molecular weight, 0.08 wt % butylated hydroxytoluene, 1 wt % black ferric oxide, and 0.25 wt % magnesium stearate; a wall surrounds the combinations of (1) (4), (2) (4), and (3) (4), said wall comprising 99 wt % cellulose acetate comprising a 39.8% acetyl content and 1 wt % polyethylene glycol of 3,350 molecular weight; and an exit passageway in the wall for providing dosage form with a start-up time of 1½ hours or less and a delivery dose of 91% or greater for treating incontinence in a patient in need of oxybutynin therapy.

### Examples 10 and 11

A dosage form for the oral administration of oxybutynin chloride is prepared comprising a drug composition consisting of 5 wt % oxybutynin chloride, 5 wt % osmotic salt, 88 wt % polyoxyethylene oxide possessing a 200,000 molecular weight, and 2 wt % binder. The dosage form exhibited a start-up time of 1.6 hours, and it delivered 88.5% of the oxybutynin chloride at a 67% zero order rate. A dosage form for the oral administration of oxybutynin chloride is prepared comprising 5 wt % oxybutynin chloride, 2.5 wt % polyoxyethylene-20-sorbitan monooleate, 10 wt % osmotic salt, 80.5 wt % polyoxyethylene oxide possessing a 200,000 molecular weight and 2 wt % binder. The dosage form exhibited a 1.6 hour start-up time, and it delivered 91.6% of the oxybutynin hydrochloride at 73% zero order rate.

### METHOD OF USING THE INVENTION

The invention pertains additionally to the use of the therapeutic composition and the use of the dosage form by providing a method for delivering oxybutynin orally to a warm-blooded animal, including a human patient, in need of oxybutynin therapy. The method comprises administering orally the therapeutic composition to a patient for oxybutynin therapy. The method also comprises: (A) admitting orally into the patient a dosage form comprising (B) a semipermeable wall that surrounds (C) a therapeutic composition comprising (C) oxybutynin. The dosage form imbibes fluid through the wall into the dosage form in response to the concentration gradient across the semipermeable wall. The therapeutic composition in the dosage form develops osmotic energy that causes the therapeutic composition to be administered through the exit (D) from the dosage form over a prolonged period of time up to 24 hours to provide sustained and controlled oxybutynin therapy. The method of the invention comprises also: (A) admitting orally into a warm-blooded animal a dosage form comprising: (B) a wall surrounding a compartment, the wall comprising a semipermeable polymeric composition permeable to the passage of fluid and substantially impermeable to the passage of oxybutynin; (C) a therapeutic composition comprising oxybutynin in the compartment (E) a hydrogel push-displacement composition in the compartment comprising an osmotic formulation for imbibing and absorbing fluid for expanding in size for pushing the therapeutic oxybutynin composition from the dosage form; and (D) at least one passageway in the wall for releasing the oxybutynin; (F) imbibing fluid through the semipermeable wall at a fluid-imbibing rate determined by the permeability of the semipermeable wall and the osmotic pressure across the semipermeable wall causing the push-displacement composition to expand; and (G) delivering the therapeutically active oxybutynin from the delivery device through the exit passageway to a warm-blooded animal over a prolonged period of time up to 24 hours. The oxybutynin is administered by the method of the invention in the therapeutic range that avoids a toxic dose and avoids an ineffective dose for antispasmodic therapy. The oxybutynin is administered to patients with uninhibited neurogenic and reflex neurogenic bladder for increased vesual capacity which diminishes the frequency of uninhibited contractions of the detrusor muscle and delays the desire to void. The dosage form is indicated for the relief of symptoms associated with voiding such as urgency, urge incontinence, frequency, nocturia and incontinence in patients in neurogenic bladder.

The therapeutic compositions and the dosage forms of this invention can be used in methods for administering oxybutynin by the oral route into the gastrointestinal tract, and for delivering oxybutynin through the sublingual and buccal routes. The sublingual and buccal routes can be used for administering a smaller dose for immediate therapy, and as a by-pass of the first pass of hepatic metabolism of oxybutynin.

In summary, it will be appreciated that the present invention contributes to the art an unobvious dosage form that possesses practical utility, can administer a drug at a dose-metered release rate per unit time. While the invention has been described and pointed out in detail with reference to operative embodiments thereof, it will be understood by those skilled in the art that various changes, modifications, substitutions and omissions can be made without departing from the spirit of the invention. It is intended, therefore, that the invention embrace those equivalents within the scope of the claims which follow.

1. A therapeutic composition comprising a member selected from the group consisting of oxybutynin and its pharmaceutically acceptable salt and a pharmaceutically acceptable surfactant for providing a sustained-release dosage form for the treatment of a patient with an overactive bladder accompanied with symptoms of urinary frequency.

2. A therapeutic composition comprising 240 mg to 650 mg of an oxybutynin selected from the group consisting of the racemate, the R-enantiomer and the S-enantiomer and a pharmaceutically acceptable surfactant, which composition is indicated for the manufacture of an oral sustained-release dosage form for the management of bladder instability associated with incontinence.

3. A therapeutic composition comprising 240 mg to 650 mg of a member selected from the group consisting of oxybutynin and its pharmaceutically acceptable salt, and a pharmaceutically acceptable surfactant selected from the group consisting of amphoteric, anionic, cationic and nonionic surfactants.

4. A therapeutic composition comprising 240 mg to 650 mg of a member selected from the group consisting of oxybutynin and its pharmaceutically acceptable salt, and 0.01 mg to 25 mg of a pharmaceutically acceptable surfactant selected from the group consisting of polyoxyethylenated sorbital monolaurate, polyoxyethylenated sorbitan monopalmitate, polyoxyethylenated sorbitan monostearate, polyoxyethylenated sorbitan tristearate, polyoxyethylenated sorbitan monooleate, polyoxyethylenated sorbitan trioleate, and polyoxyethylenated stearic acid.

5. A therapeutic composition comprising a member selected from the group consisting of 240 mg to 650 mg of oxybutynin, 0.01 mg to 25 mg of a surfactant, and 10 mg to 250 mg of a pharmaceutically acceptable hydrophilic polymer.

6. A therapeutic composition comprising 240 mg to 650 mg of oxybutynin, 0.01 to 25 mg of a surfactant, and 10 mg to 250 of a polyalkylene oxide.

7. A therapeutic composition comprising 240 mg to 650 mg of oxybutynin, 0.01 to 25 mg of a surfactant, and 0.5 to 50 mg a hydroxypropylalkylcellulose

8. A method for the management of bladder instability associated with incontinence in a patient, wherein the method comprises administering an oral sustained-release dosage form comprising oxybutynin and a surfactant for the management over a sustained-release period up to twenty-four hours.

9. A method for the treatment of a patient with an overactive bladder accompanied with symptons of urinary frequency, wherein the method comprises admitting orally into the patient a sustained-release dosage from comprising oxybutynin, a surfactant, and a binder that imparts cohesiveness to the composition, for providing sustained-release oxybutynin therapy up to twenty-four hours.

10. A method for treating a patient with urge incontinence, wherein the method comprises administering orally to the patient a sustained-release dosage form comprising oxybutynin, a surfactant and a hydroxypropylalkylcellulose that is administered over a prolonged period for treating the patient.

11. A method for treating a patient with urge incontinence, wherein the method comprises administering orally to the patient a sustained-release dosage form comprising oxybutynin, a surfactant, and a hydrophilic polymer that is administered over a prolonged period up to twenty-four hours for treating the patient.

12. A method for providing antispasmodic therapy in a patient with uninhibited neurogenic and reflex neurogenic bladder, wherein the method comprises administering to the patient a sustained-release dosage form comprising the antispasmodic oxybutynin in a therapeutic range that avoids a toxic dose and avoids an ineffective dose over a prolonged time up to twenty-four hours for providing antispasmodic therapy in the patient with uninhibited neurogenic and reflex neurogenic bladder.

13. A method for providing antispasmodic therapy in a patient with uninhibited neurogenic and reflex neurogenic bladder, wherein the method comprises administering to the patient a composition comprising the antispasmodic oxybutynin and means for enhancing the administration of the antispasmodic oxybutynin over a prolonged time for providing the antispasmodic therapy.

14. A dosage form comprising: a wall; a therapeutic composition comprising oxybutynin and a surfactant; and means in the dosage form for delivering the composition from the dosage form.

15. A dosage form comprising: a therapeutic composition comprising oxybutynin, a surfactant, and a polyalkylene oxide; a semipermeable wall that surrounds the therapeutic composition; and, an exit in the dosage form for delivering the oxybutynin from the dosage form.

16. A dosage form comprising: a therapeutic composition comprising oxybutynin, a surfactant, and a hydrophilic polymer; a wall at least in part permeable to the passage of fluid that surrounds the therapeutic composition; and, an exit in the dosage form for the release of oxybutynin from the dosage form.

17. A dosage form comprising: a therapeutic composition comprising oxybutynin and a surfactant; an expandable composition comprising a hydrophilic member; a wall in at least a part permeable to the passage of fluid that envelops the therapeutic and the expandable compositions; and an exit in the dosage form for releasing the oxybutynin from the dosage form.

18. The dosage form according to claim 17, wherein the therapeutic composition comprises an osmotically effective solute.

19. The dosage form according to claim 17, wherein the therapeutic composition comprises a hydrophilic polymer.

20. The dosage form according to claim 17, wherein the therapeutic composition comprises a polyalkylene oxide.

21. The dosage form according to claim 17, wherein the surfactant is a member selected from the group consisting of an amphoteric, anionic, cationic, and nonionic surfactant.

22. The dosage form according to claim 17, wherein the expandable composition comprises an osmotically active solute.

23. The dosage form according to claim 17, wherein hydrophilic member in the expandable composition is a hydrogel.

24. The dosage form according to claim 17, wherein the hydrophilic member in the expandable composition is polyethylene oxide.

25. The dosage form according to claim 17, wherein the surfactant is selected from the group consisting of polyoxyethylenated sorbitol monolaurate, polyoxyethylenated sorbitan monopalmitate, polyoxyethylenated sorbitan monostearate, polyoxyethylenated sorbitan tristearate, polyoxyethyleneated sorbitan monooleate, polyoxyethylenated sorbitan trioleate, and polyoxyethylenated stearic acid.
